(19) United States
(12) Patent Application Publication
(10) Pub. No.: US 2003/0224416 A1
(43) Pub. Date: Dec. 4, 2003

(54) Schizophrenia related gene

(76) Inventors: Susan Cochran, Glasgow (GB); Keiji Yamagami, Tokyo (JP); Yoshitaka Ohashi, Tokyo (JP)

Correspondence Address: WENDEROTH, LIND & PONACK, L.L.P., 2033 K STREET N. W., SUITE 800, WASHINGTON, DC, 20006-1021, US

(21) Appl. No.: 10/400,435

(22) Filed: Mar. 28, 2003

(30) Foreign Application Priority Data
Mar. 28, 2002 (EP) 02007114.8

(51) Int. Cl. C12Q 1/68; G01N 33/53; G01N 33/567

(52) U.S. Cl. 435/6; 435/7.2

(57) ABSTRACT

The present invention provides a new use of the polynucleotide of SEQ ID NO:1, NO:3 and NO:5 or fragments thereof, or the polypeptide encoded from that polynucleotide in the field of the schizophrenia.

### TECHNICAL FIELD OF THE INVENTION

The present invention relates to the gene of the voltage-gated potassium channels Kv3. The present invention also relates to its use in the therapy and/or diagnosis of schizophrenia and to a method of screening for a compound which modulates the expression of the gene or the activity of the voltage-gated-potassium channels.

### BACKGROUND OF THE INVENTION

Schizophrenia is a devastating mental illness that affects 1%,of the world population, the aetiology of which remains elusive. To date, there is a poor understanding of the genes involved. One of the goals of modern antipsychotic drug development is to produce a drug which is more effective in ameliorating the negative symptoms and cognitive deficits characteristic of schizophrenia than existing therapies. Although typical and a typical antipsychotic drugs, such as haloperidol and clozapine respectively, are effective in attenuating the positive symptoms, they are ineffective (haloperidol) or minimally effective (clozapine) against the negative symptoms and cognitive dysfunction associated with the disease (Goldberg, T. E. et al. British Journal of Psychiatry, 1993, 162; 43-48). The development of improved antipsychotic drugs that will have superior action against the negative symptoms and cognitive dysfunction has been severely hampered by the lack of knowledge of which genes are involved and/or associated with schizophrenia. Moreover little is known about the genes, or more specifically any alteration of expression or mutation of the genes in a patient suffering from schizophrenia.

The Shaw-related family of voltage-gated potassium channels (also known as Kv3 channels) are encoded by four genes (Kv3.1, Kv3.2, Kv3.3 and Kv3.4). The products of these genes have differential expression within the mammalian brain, with Kv3.1 and Kv3.3 channels being predominantly located within a subset of GABAergic interneurons within the brain, namely parvalbumin-containing interneurons (Weiser et al., The Journal of Neuroscience, March 1994, 14(3); 949-972). The function of Kv3.1 and Kv3.3 channels within these interneurons is to allow fast repolarisation of the cells and thus, contributes to the fast-firing phenotype of these neurons (Du et al. The Journal of Neuroscience, Jan. 15, 1996, 16(2); 506-518, Chow et al., The Journal of Neuroscience, Nov. 1, 1999, 19(21); 9332-9345; Erisir et al., The Journal of Neurophysiology, 1999, 82; 2476-2489). Kv3.2 expression is predominantly (90%) within thalamic relay neurons throughout the dorsal thalamus and also in parvalbumin-containing and other GABAergic (Marinotti) interneurons (Moreno et al., The Journal of Neuroscience, August, 1995,15(8); 5486-5501). Parvalbumin expression has been shown to be altered within the prefrontal cortex in schizophrenia (Beasley and Reynolds, Schizophrenia Research, 1997, 24; 349-355), however Kv3.1, Kv3.2 and Kv3.3 expression as not been investigated in this disease state.

The Shaw-related voltage-gated potassium channels are known to have many names (see below).

| Present Nomenclature | Splice Variants | |
|---|---|---|
| (Alternate names) | (Alternate names) | Gene |
| Kv3.1 | Kv3.1a | KCNC1 |
| (KShIIIB; NGK2-KV4) | (NGK2; KShIIIB) | |
| | Kv3.1b | |
| | (KV4) | |
| Kv3.2 | Kv3.2a | KCNC2 |
| (KShIIIA) | (RKShIIIA; KShIIIA.1) | |
| | Kv3.2b | |
| | (KShIIIA.3) | |
| | Kv3.2d | |
| | (KShIIIA.2) | |
| Kv3.3 | Kv3.3a | KCNC3 |
| (KShIIID) | (KShIIID.1) | |
| | Kv3.3b | |
| | (KShIIID.2) | |

Table adapted from Weiser et al., The Journal of Neuroscience, March 1994, 14(3); 949-972.

In the present specification, the inventors use the name Kv3.1 for the voltage-gated potassium channel 3.1; Kv3.2 for the voltage-gated potassium channel 3.2 and Kv3.3 for the voltage-gated potassium channel 3.3. Alternate splice variants of each of the Kv3 genes given in the table above), with the splice variants for each gene differing only in their intracellular C-terminal sequence (Coetzee et al., Annals of the New York Academy of Sciences, 1999, 868; 233-285.

The human Kv3 genes have also been cloned. Kv3.1 α-subunit: GenBank accession number NM_{—}004976; Localization of a highly conserved human potassium channel gene (NGK2-Kv4; KCNCl) to chromosome 11p15. Reid T; Rudy B; Vega-Saenz de Miera E; Lau D; Ward D C and Sen K. (1993). Genomics 15 (2), 405-411. The Kv3.1a α-subunit displays ˜99% sequence identity to the rat sequence, and there is >90% sequence identity between rat and mouse sequences. Kv3.2: GenBank accession number AY118169. Kv3.3:GenBank accession number NM_{—}004977; Genomic organization, nucleotide sequence and cellular distribution of a Shaw-related potassium channel gene, Kv3.3, and mapping of Kv3.3 and Kv3.4 to human chromosomes 19 and 1. Ghanshani et al., Genomics 12(2) 1992; 190-196)

Chronic treatment with phencyclidine (hereinafter PCP) in the rat provides an animal model of schizophrenia with superior face, construct and predictive validity (WO01/75440). It has been shown to mimic metabolic changes (metabolic hypofunction in the prefrontal cortex, thalamus and auditory structures) that are observed in schizophrenic patients, that can be correlated to both positive and negative symptomology and cognitive deficits associated with the disease, and also neurochemical changes observed in postmortem tissue from schizophrenic patients such as decreased parvalbumin expression and decreased 5HT2A binding. Parvalbumin mRNA was selectively decreased within certain brain regions in the chronic PCP rat model of schizophrenia, the prelimbic region of the prefrontal cortex and the reticular nucleus of the thalamus and this effect was modulated by chronic antipsychotic treatment. Due to the high degree of co-localization between parvalbumin and the Kv3.1 and Kv3.3 channels, the aim was to determine whether Kv3.1 α-subunit expression is similarly altered by the same chronic PCP rat model of schizophrenia. Also, due to the localization of Kv3.2 channels in thalamic relay neurons and GABAergic interneurons, it was also of interest whether Kv3.2 mRNA expression was also altered by the chronic PCP rat model of schizophrenia.

### SUMMARY OF THE INVENTION

The present inventors have found that the Kv3.1 α-subunit ene, or a nucleotide having SEQ ID NO:1; the Kv3.2 gene, or nucleotide having SEQ ID NO:3; and Kv3.3 gene, or a nucleotide having SEQ ID NO:5 show, altered expression in the brain of the chronic PCP rat model of schizophrenia, and also found that the nucleotides and the protein that these nucleotides encodes are useful for the diagnosis or treatment of schizophrenia. The present invention also provides a method of screening a compound that regulates expression and activity of this nucleotide and/or protein. Accordingly, the present invention provides the following.

(1) A method for diagnosing schizophrenia, which comprises use of at least one kind of polynucleotide selected from the group consisting of a polynucleotide having SEQ ID NO:1, a polynucleotide having SEQ ID NO:3, a polynucleotide having SEQ ID NO:5, a fragment thereof, and a polynucleotide having a sequence complementary to such sequence, as an index of schizophrenia.

(2) The method of the above-mentioned (1), wherein the at least one kind of polynucleotide is selected from the group consisting of a polynucleotide having SEQ ID NO:1, a polynucleotide having SEQ ID NO:3, a polynucleotide having SEQ ID NO:5 and a polynucleotide having a sequence complementary to such sequence.

(3) The method of the above-mentioned (1), wherein the at least one kind of polynucleotide is a polynucleotide having SEQ ID NO:1.

(4) A method for diagnosing schizophrenia, which comprises use of at least one kind of polypeptide selected from the group consisting of a polypeptide encoded by the polynucleotide of SEQ ID NO:1, a polypeptide encoded by the polynucleotide of SEQ ID NO:3, a polypeptide encoded by the polynucleotide of SEQ ID NO:5, and a fragment thereof, as an index of schizophrenia.

(5) The method of the above-mentioned (4), wherein the at least one kind of polypeptide is selected from the group consisting of a polypeptide encoded by the polynucleotide of SEQ ID NO:1, a polypeptide encoded by the polynucleotide of SEQ ID NO:3 and a polypeptide encoded by the polynucleotide of SEQ ID NO:5.

(6) The method of the above-mentioned (4), wherein the at least one kind of polypeptide is a polypeptide encoded by the polynucleotide of SEQ ID NO:1.

(7) A method for identifying a compound modulating the activity of Kv3 channels, which comprises analyzing an expression of Kv3 channel using at least one kind of polynucleotide selected from the group consisting of a polynucleotide having SEQ ID NO:1, a polynucleotide having SEQ ID NO:3, a polynucleotide having SEQ ID NO:5, a fragment thereof and a polynucleotide having a sequence complementary 5 to such sequence.

(8) The method of the above-mentioned (7), wherein the expression of Kv3 channel is analyzed using at least one kind of polynucleotide selected from the group consisting of a polynucleotide having SEQ ID NO:1, a polynucleotide having SEQ ID NO:3, a polynucleotide having SEQ ID NO:5 and a polynucleotide having a sequence complementary to such sequence.

(9) The method of the above-mentioned (7), wherein the expression of Kv3 channel is analyzed using a polynucleotide having SEQ ID NO:1.

(10) A method for identifying a compound modulating the activity of Kv3 channels, which comprises analyzing an expression of Kv3 channel using at least one kind of polypeptide selected from the group consisting of a polypeptide encoded by the polynucleotide of SEQ ID NO:1, a polypeptide encoded by the polynucleotide of SEQ ID NO:3, a polypeptide encoded by the polynucleotide of SEQ ID NO:5, and a fragment thereof.

(11) The method of the above-mentioned (10), wherein the expression of Kv3 channels is analyzed using at least one kind of polypeptide selected from the group consisting of a polypeptide encoded by the polynucleotide of SEQ ID NO:1, a polypeptide encoded by the polynucleotide of SEQ ID NO:3, and a polypeptide encoded by the polynucleotide of SEQ ID NO:5.

(12) The method of the above-mentioned (10), wherein the expression of Kv3 channel is analyzed using a polypeptide encoded by the polynucleotide of SEQ ID NO:1.

(13) A method for screening a compound regulating the expression of at least one kind of polynucleotide selected from the group consisting of a polynucleotide having SEQ ID NO:1, a polynucleotide having SEQ ID NO:3 and a polynucleotide having SEQ ID NO:5, which comprises the steps of

(a) bringing a test compound into contact with a cell (as used herein, the cell is capable of expression of polynucleotide, which is at least one kind of polynucleotide selected from the group consisting of a polynucleotide having SEQ ID NO:1, a polynucleotide having SEQ ID NO:3, a polynucleotide having SEQ ID NO:5 and a fragment thereof, and whose expression is desired to be controlled;

(b) detecting the expression of polypeptide encoded by the polynucleotide in the cell; and

(c) determining a compound promoting or suppressing the expression of the polypeptide as compared to a control (administration of vehicle alone).

(14) A method for screening a compound regulating the, expression of a polynucleotide having SEQ ID NO:1, which comprises the steps of:

(a) bringing a test compound into contact with a cell (as used herein, the cell is capable of expression of a polynucleotide having SEQ ID NO:1);

(b) detecting the expression of polypeptide encoded by the polynucleotide in the cell; and

(c) determining a compound promoting or suppressing the expression of the polypeptide as compared to a control (administration of vehicle alone).

(15) A method for screening a compound regulating the expression of at least one kind of polynucleotide selected from the group consisting of a polynucleotide having SEQ ID NO:1, a polynucleotide having SEQ ID NO:3 and a polynucleotide having SEQ ID NO:5, which comprises the steps of:

(a) bringing a test compound into contact with a cell (as used herein, the cell is capable of expression of polynucleotide, which is at least one kind of polynucleotide selected from the group consisting of a polynucleotide having SEQ ID NO:1, a polynucleotide having SEQ ID NO:3, and a polynucleotide having SEQ ID NO:5, and whose expression is desired to be controlled);

(b) detecting the expression of polynucleotide in the cell; and

(c) determining a compound promoting or suppressing the expression of the polynucleotide as compared to a control (administration of vehicle alone).

(16) A method for screening a compound regulating the expression of a polynucleotide having SEQ ID NO:1, which comprises the steps of:

(a) bringing a test compound into contact with a cell (as used herein, the cell is capable of expression of polynucleotide having SEQ ID NO:1);

(b) detecting the expression of polynucleotide in the cell; and

(c) determining a compound promoting or suppressing the expression of the polynucleotide as compared to a control (administration of vehicle alone).

### BRIEF DESCRIPTION OF THE DRAWING

FIG. 1 shows a chronic intermittent PCP treatment regime (YRING Model).

### DETAILED DESCRIPTION OF THE INVENTION

The present invention relates to methods for prognostic and/or diagnostic evaluation of schizophrenia and/or for the identification of subjects who are predisposed to schizophrenia, for example by examination of allelic variation of the gene identified herein in an individual or by determination of the expression of the gene in an individual. Furthermore, the invention provides methods for evaluating the efficacy of drugs for such disorders, and monitoring the progress of patient symptoms involved in clinical trials for the treatment of such disorders.

The invention further provides methods for the identification of compounds that modulate the expression of the Kv3 genes and/or the activity of the Kv3.1, Kv3.2 or Kv3.3 channels. Such identified compounds may be used in the treatment of schizophrenia.

By the “regulate” is meant here the action to promote or suppress the expression of the gene, which varies according to the desired effect. In consideration of the application to Schizophrenia, however, screening of a compound that increases the level of expression of Kv3.1 α-subunit gene is preferable and a compound that decreases the level of the Kv3.2 and Kv3.3 gene is preferable.

In the present invention, the “polynucleotide having a sequence complementary” means a sequence complementary to a target sequence to the extent that hybridization under stringent conditions occurs, wherein stringency can be appropriately determined according to the method employed.

In the present invention, the “fragment” means a poly(oligo)nucleotide or a poly(oligo)peptide obtained by fragmentation of polynucleotide having SEQ ID NO:1, a polynucleotide having SEQ ID NO:3, a polynucleotide having SEQ ID NO:5, a polypeptide encoded by the polynucleotide of SEQ ID NO:1 (i.e., SEQ ID NO:2), a polypeptide encoded by the polynucleotide of SEQ ID NO:3 (i.e., SEQ ID NO:4) or a polypeptide encoded by the polynucleotide of SEQ ID NO:5 (i.e., SEQ ID NO:6) to the extent that affords reproduction of characteristic mode of expression found in the pharmacology of schizophrenia. For example, the fragment may be any as long as the expression of a polynucleotide having SEQ ID NO:1, a polynucleotide having SEQ ID NO:3, a polynucleotide having SEQ ID NO:5, a polypeptide having SEQ ID NO:2, a polypeptide having SEQ ID NO:4 and a polypeptide having SEQ ID NO:6 in patients with schizophrenia can be observed and the size thereof is not particularly limited.

The “at least one kind of polynucleotide” of the “at least one kind of polynucleotide selected from the group consisting of a polynucleotide having SEQ ID NO:1, a polynucleotide having SEQ ID NO:3, a polynucleotide having SEQ ID NO:5, a fragment thereof, and a polynucleotide having a sequence complementary to the sequence” intends, in addition to each polynucleotide, two kinds of polynucleotides selected from the group, or 3 kinds of polynucleotides selected from the group and the like. It means, for example, one kind of, two kinds of and all three kinds of polynucleotides selected from the group consisting of a polynucleotide having SEQ ID NO:1, a polynucleotide having SEQ ID NO:3 and a polynucleotide having SEQ ID NO:5. Likewise, the “at least one kind of polypeptide” of the “at least one kind of polypeptide selected from the group consisting of a polypeptide encoded by the polynucleotide of SEQ ID NO:1, a polypeptide encoded by the polynucleotide of SEQ ID NO:3, a polypeptide encoded by the polynucleotide of SEQ ID NO:5, a fragment thereof, and a polypeptide having a sequence complementary to the sequence” intends, in addition to each polypeptide, two kinds of polypeptides selected from the group, or 3 kinds of polypeptides selected from the group and the like. It means, for example, one kind of, two kinds of and all three kinds of polypeptide selected from the group consisting of a polypeptide encoded by the polynucleotide of SEQ ID NO:1, a polypeptide encoded by the polynucleotide of SEQ ID NO:3 and a polypeptide encoded by the polynucleotide of SEQ ID NO:5. Preferably, it is a polynucleotide having SEQ ID NO:1 or polypeptide encoded by the polynucleotide of SEQ ID NO:1.

The present invention provides a method for screening a compound regulating the expression of at least one kind of polynucleotide selected from the group consisting of a polynucleotide having SEQ ID NO:1, a polynucleotide having SEQ ID NO:3 and a polynucleotide having SEQ ID NO:5, which comprises the steps of

(a) bringing a test compound into contact with a cell (as used herein, the cell is capable of expression of polynucleotide, which is at least one kind of polynucleotide selected from the group consisting of a polynucleotide having SEQ ID NO:1, a polynucleotide having SEQ ID NO:3, a polynucleotide having SEQ ID NO:5 and a fragment thereof and whose expression is desired to be controlled);

(b) detecting the expression of polypeptide encoded by the polynucleotide in the cell; and

(c) determining a compound promoting or suppressing the expression of the polypeptide as compared to a control (administration of vehicle alone).

In the above-mentioned screening method, the “test compound” is free of any particular limitation as long as it is the object compound for which capability of regulating the expression of at least one kind of polynucleotide selected from the group consisting of a polynucleotide having SEQ ID NO:1, a polynucleotide having SEQ ID NO:3 and a polynucleotide having SEQ ID NO:5, preferably a polynucleotide having SEQ ID NO:1 is desired to be determined, and it may be a novel or known compound. A combination drug can be the object compound. More specifically, chronic PCP administration that affects the expression of the Kv3 mRNAs is combined with administration of the test compound. By analyzing the metabolic and neurochemical state induced by the chronic PCP administration and changes in the expression of the Kv3 mRNAs, the effect of the test compound can be investigated.

In the above-mentioned screening method, the cell is free of any particular limitation as long as it expresses at least one kind of polynucleotide selected from the group consisting of a polynucleotide having SEQ ID NO:1, a polynucleotide having SEQ ID NO:3 and a polynucleotide having SEQ ID NO:5, preferably a polynucleotide having SEQ ID NO:1. It may be naturally occurring or processed to express said polynucleotide by genetic recombination.

For detection of intracellular expression of polypeptide and comparison with control, the methods and techniques generally used in this field can be used and those of ordinary skill in the art can modify them suitably. Specific examples thereof include western blotting capable of detection at a protein level.

Moreover, the method for screening a compound regulating the expression of at least one kind of polynucleotide selected from the group consisting of a polynucleotide having SEQ ID NO:1, a polynucleotide having SEQ ID NO:3 and a polynucleotide having SEQ ID NO:5, may comprise the steps of

(a) bringing a test compound into contact with a cell (as used herein, the cell is capable of expression of polynucleotide, which is at least one kind of polynucleotide selected from the group consisting of a polynucleotide having SEQ ID NO:1, a polynucleotide having SEQ ID NO:3 and a polynucleotide having SEQ ID NO:5 and whose expression is desired to be controlled);

(b) detecting the expression of polynucleotide in the cell; and

(c) determining a compound promoting or suppressing the expression of the polynucleotide as compared to a control (administration of vehicle alone).

Each term is as mentioned above, and for detection of intracellular expression of polynucleotide and comparison with control, the methods and techniques generally used in this field can be used and those of ordinary skill in the art can modify them suitably. Specific examples thereof include northern blotting capable of detection at an mRNA level.

The information obtained through the screening method and diagnostic method of the present invention (hereinafter to be generally referred to as characterization) can suggest relevant methods for the treatment or control of schizophrenia. For example, relevant treatment can include a regulation of gene expression and/or a modulation of gene product activity. Characterization procedures such as those described herein can indicate whether such regulation/modulation should be positive or negative. As used herein, “positive regulation/modulation” refers to an increase in gene expression and/or the activity of gene product of interest. “Negative regulation/modulation”, as used herein, refers to a decrease in gene expression and/or activity.

In vitro systems can be designed to identify compounds capable of binding with said Kv3 genes products of the invention.

SEQ ID NO:1 shows the nucleotide sequence of the rat Kv3.1 α-subunit gene of the present invention, SEQ ID NO:3 shows the nucleotide sequence of the rat Kv3.2 gene of the present invention and SEQ ID NO:5 shows the nucleotide sequence of the rat Kv3.3 gene of the present invention, which were observed to be differentially expressed in the brain in the chronic PCP rat model of schizophrenia.

SEQ ID NO:2 shows the amino acid sequence of the rat Kv3.1 α-subunit of the present invention, SEQ ID NO:4 shows the amino acid sequence of the rat Kv3.2 of the present invention and SEQ ID NO:6 shows the amino acid sequence of the rat Kv3.3 of the present invention which were observed to be differentially expressed in the brain in the chronic PCP rat model of schizophrenia.

SEQ ID NO:7 shows the oligonucleotide (45mer) used for in situ hybridization for the Kv3.1 α-subunit mRNA; SEQ ID NO:8 shows the oligonucleotide (45mer) used for in situ hybridization for the Kv3.2 mRNA; SEQ ID NO:9 shows the oligonucleotide (45mer) used for in situ hybridization for the Kv3.3 mRNA.

The present invention is explained in more detail in the following by way of Experiments, which are not to be construed as limitative.

In this specification, Y-931 means 8-fluoro-12-(4-methylpiperazin-1-yl)-6H-[l]benzothieno[2,3-b][1,5]benzo-diazepine monomaleate, which is known as an a typical antipsychotic drug (WO99/11647).

Experiment

Method

Experiment 1

Male Sprague-Dawley rats, weighing 224-260 g at the beginning of the study, were randomly assigned to one of ten treatment groups as follows: vehicle (0.02N HCl in saline)-vehicle (polyethyleneglycol); PCP-vehicle; PCP-Y-931 0.3 mg/kg/day; PCP-Y-931 1 mg/kg/day; PCP-Y-931 3 mg/kg/day; PCP-Y-931 10 mg/kg/day; PCP-olanzapine 0.3 mg/kg/day; PCP-olanzapine 1 mg/kg/day; PCP-olanzapine 3 mg/kg/day; PCP-olanzapine 10 mg/kg/day. The dose of PCP used was 2.24 mg/kg (free base equivalent, equal to 2.58 mg/kg PCP.HCl) and PCP was administered according to the chronic PCP rat model of schizophrenia (WO01/75440). Briefly, i.p. injections of PCP (2.24 mg/kg) or saline (1 ml/kg) were administered once daily on days 1-5, 8, 10, 12, 15, 17, 19, 22, 24, 26. Osmotic minipumps (2ML4 (28 day), Alzet) were implanted subcutaneously on day 8 prior to PCP injection to deliver doses of antipsychotics or vehicle detailed above for 21 days of drug treatment. The osmotic minipumps were primed in vitro for 24 hours in order to ensure pumping started immediately upon implantation.

29 days after the first PCP injection, the animals were sacrificed, the brains removed and stored at −70° C. Osmotic minipumps were also removed and the dose administered was verified. The brains were sectioned at −20° C. using a cryostat (Leica, CM1850), with 14 μm coronal sections being thaw-mounted onto silanized slides (Dako, Japan) from the following bregma levels according to the Rat Brain Atlas, Paxinos and Watson (2000): 3.22 mm; 1.6 mm, −1.80 mm and −4.8 mm. The sections were then stored at −80° C. over silica gel until required. The oligonucleotide (45mer) used for in situ hybridization for the Kv3.1 α-subunit mRNA was 5′-ATAGTCGAAGTGGCTGTGGGCGTCCGGCTCCGCCAGCCAGGCAAG-3′ (SEQ ID NO:7) complementary to bases 1261-1305 of the rat Kv3.1 α-subunit (GenBank Accession Number NM_{—}012856; Luneau et al., Proc. Natl. Acad. Sci. USA. 1991, 88; 3932-3936). This probe sequence recognizes both alternate splice variants (Kv3.1a and Kv3.1b) of the Kv3.1 channel. The probe was 3′ end-labeled with terminal deoxyribonucleotidyl transferase (Boehringer Mannheim) and with the isotope 5-α-³⁵S-dATP (Amersham, specific activity >1000 Ci/mmol) and incubated at 37° C. for one hour. The reaction was terminated by addition of 40 μl diethyl pyrocarbonate (DEPC)-treated water. The probe was purified using the QIAquick nucleotide removal kit (Qiagen). The extent of probe labeling was assessed using scintillation counting, and probes labeled from 100,000 to 300,000 d.p.m. μl⁻¹ were used for in situ hybridization. On the day of the in situ hybridization experiment the sections were fixed in 4% paraformaldehyde-0.1M phosphate buffer for 30 mins at 4° C., rinsed twice for 5 mins in 0.1M phosphate buffer saline (PBS), acetylated with 0.25% acetic anhydride in 0.1M triethanolamine for 10 mins, rinsed twice for 5 mins in 2×SSC (0.15M NaCl, 15 mM sodium citrate), dehydrated and delipidated through a graded series of ethanol and chloroform. The sections were then air dried and then hybridized overnight at 42° C. with 0.2-1×10⁻⁶ d.p.m. of the labeled probe in 100 μl of hybridization solution (Hybrisol 1, Intergen) containing 0.1M dithiothreitol. After hybridization, the sections were washed twice for 10 secs in 1×SSC at 55° C., four times for 15 mins in 1×SSC at 55° C., for 1 hour in 1×SSC at room temperature and twice in water for 5 mins. The sections were then dipped successively in 60%, 80%, 90%, 95% and 100% ethanol and air dried. The sections were exposed to BAS 5000 image plates and developed after 7 days. Kv3.1 α-subunit mRNA expression was quantified using a computer-based densitometry system (MCID 5, Canada). Statistical analysis of the data was performed using a students t-test (vehicle-vehicle vs PCP-vehicle) followed by Dunnett's Method (PCP-vehicle vs PCP-antipsychotics). Statistical significance was defined as p<0.05.

Experiment 2

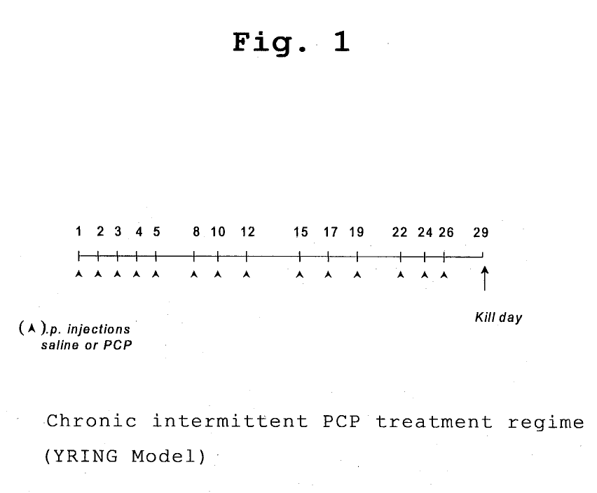

In a second experiment using different method for in situ hybridisation, the effect of chronic PCP treatment on Kv3.1, Kv3.2 and Kv3.3 mRNA were examined. 20 male hooded Long Evans rats (250-300 g at the beginning of the study, Harlan UK) were randomly assigned to either vehicle (saline, n=10) or PCP (2.58 mg/kg PCP.HCl, n=10) treatment groups. Animals were treated according to the YRING model (FIG. 1) and killed by cervical dislocation 72 hours after the last exposure to PCP. The brains were stored at −70° C. until required.

In Situ Hybridisation

20 μm coronal sections were cut on a cryostat at −20° C. and collected onto poly-L-lysine coated slides. The sections were fixed, dehydrated and stored under ethanol at 4° C. until required. 45mer oligonucleotide probes designed against rat mRNA for parvalbumin (GeneBank Accession number NM_{—}022499, bases 223-267), Kv3.1 (GeneBank Accession number NM_{—}012856, bases 1261-1305; SEQ ID NO: 7), Kv3.2 (GeneBank Accession number NM_{—}139217, bases 1021-1065; SEQ ID NO: 8) and Kv3.3 (GeneBank Accession number NM 053997, bases 241-285; SEQ ID 30 NO: 9) were 3′ end-labeled with terminal deoxyribonucleotidyl transferase and ³⁵Sd-ATP. On the day of in situ hybridization, the sections from the chronic study were allowed to dry at room temperature then hybridized with the appropriate probe overnight at 42° C. The sections were then washed, dehydrated and air-dried before being exposed to x-ray film for 7-10 days.

The quantification of mRNA expression was carried out using computer-based densitometry (MCID 5). Bilateral relative optical density measurements were taken from duplicate sections from each animal (n=8-10 per treatment group for the chronic study). The results were analyzed using individual t-tests for each brain region. Statistical significance was defined as p<0.05, again after correction for multiple comparisons using the sequential Bonferroni correction factor.

Results

Experiment 1

Table 1 shows the effect of chronic PCP and antipsychotic treatment on Kv3.1 α-subunit mRNA expression. Chronic PCP treatment selectively decreased Kv3.1 α-subunit mRNA expression within the prelimbic region of the prefrontal cortex (PrL, −31%), the dorsal part of the reticular nucleus of the thalamus (dRt, −15%) and the dorsolateral striatum (DLStr, −16%). Within the PrL, both Y-931 and olanzapine, at all doses administered, significantly reversed the PCP-induced decrease back to control levels. However, within the dRt and the DLStr, neither Y-931 nor olanzapine significantly modulated the PCP-induced effect.

Compared to PCP-vehicle, PCP-olanzapine treatment (1 mg/kg/day, only) produced a significant decrease in Kv3.1 α-subunit mRNA expression within the granule cell layer of the dentate gyrus (DGgcl) and a significant increase in Kv3.1 α-subunit mRNA expression within the ventromedial striatum (VMStr) (0.3 & 3 mg/kg/day olanzapine, only).

Experiment 2

Table 2 shows the levels of Kv3.1 mRNA expression within the rat brain after chronic intermittent treatment according to the YRING model. Very selective changes in Kv3.1 mRNA expression were observed after chronic intermittent exposure to PCP. A significant decrease in Kv3.1 mRNA expression was observed within the prelimbic cortex (−23%) and within the ventral part of the reticular thalamic nucleus (−19%). No other changes were observed in any other regions.

Table 3 shows the levels of Kv3.2 mRNA expression within the rat brain after chronic intermittent treatment according to the YRING model. A significant increase in Kv3.2 mRNA expression was observed within the prelimbic cortex (+16%) 1 and the primary motor cortex (+13%). A small but significant decrease in Kv3.2 mRNA expression was also observed within the anteromedial thalamic nucleus (−9%). No significant changes were observed after chronic intermittent PCP treatment within any other regions.

Table 3 shows the levels of Kv3.3 mRNA expression within the rat brain after chronic intermittent treatment according to the YRING model. Within cortical regions, an increase in Kv3.3 mRNA expression after chronic intermittent exposure to PCP was observed within the prelimbic cortex (+18%) and the ventral orbital cortex (+14%). A significant increase in Kv3.3 mRNA expression was also observed within the anterodorsal thalamic nucleus (+21%) and the ventral part of the reticular thalamic nucleus (+33%). No other changes were observed in any other regions.

TABLE 1

| | | | PCP-Y-931 | | | | PCP-Olanzapine | | | |
|---|---|---|---|---|---|---|---|---|---|---|
| | | | 0.3 | 1 | 3 | 10 | 0.3 | 1 | 3 | 10 |
| Region | Veh-Veh | PCP-Veh | mg/kg/day | mg/kg/day | mg/kg/day | mg/kg/day | mg/kg/day | mg/kg/day | mg/kg/day | mg/kg/day |
| PrL | 14.22 ± 0.21 | 9.83 ± | 12.76 ± | 12.17 ± | 12.93 ± | 12.55 ± | 12.50 ± | 12.78 ± | 13.18 ± | 12.66 ± |
| | | 0.23** | 0.62†† | 0.85† | 0.33†† | 0.67† | 0.92†† | 0.54†† | 0.43†† | 0.49†† |
| IL | 12.30 ± 0.58 | 12.28 ± | 11.64 ± 0.40 | 11.33 ± 0.60 | 12.91 ± 0.38 | 11.55 ± 0.45 | 12.00 ± 0.65 | 11.27 ± 1.08 | 11.40 ± 0.41 | 12.15 ± 0.62 |
| | | 0.82 | | | | | | | | |
| M1 | 16.68 ± 1.06 | 15.88 ± | 15.33 ± 0.38 | 14.81 ± 0.80 | 17.01 ± 0.75 | 16.51 ± 0.67 | 15.83 ± 0.69 | 16.17 ± 1.27 | 16.17 ± 0.79 | 16.27 ± 0.37 |
| | | 1.53 | | | | | | | | |
| M2 | 14.49 ± 0.65 | 14.38 ± | 13.23 ± 0.29 | 13.10 ± 0.57 | 14.25 ± 0.81 | 13.04 ± 0.53 | 13.72 ± 0.78 | 12.57 ± 0.94 | 14.48 ± 0.90 | 13.14 ± 0.99 |
| | | 0.89 | | | | | | | | |
| VO | 18.42 ± 0.99 | 19.85 ± | 19.12 ± 0.74 | 17.81 ± 1.08 | 19.32 ± 0.63 | 18.51 ± 1.08 | 19.03 ± 1.00 | 18.35 ± 1.04 | 18.96 ± 0.86 | 19.88 ± 0.58 |
| | | 0.85 | | | | | | | | |
| IO | 16.05 ± 0.96 | 16.48 ± | 17.52 ± 0.95 | 15.49 ± 0.30 | 17.52 ± 0.36 | 15.30 ± 0.62 | 15.50 ± 0.51 | 16.18 ± 0.32 | 16.83 ± 0.79 | 17.74 ± 0.37 |
| | | 0.69 | | | | | | | | |
| ACg | 14.97 ± 0.84 | 15.01 ± | 15.36 ± 0.29 | 15.10 ± 0.92 | 15.40 ± 0.83 | 14.82 ± 0.74 | 15.83 ± 0.73 | 14.83 ± 1.00 | 16.31 ± 0.40 | 15.03 ± 0.72 |
| | | 0.54 | | | | | | | | |
| Pir | 37.64 ± 1.18 | 40.65 ± | 38.95 ± 2.38 | 43.16 ± 2.76 | 38.15 ± 3.31 | 41.69 ± 1.90 | 37.73 ± 1.18 | 36.82 ± 0.51 | 43.78 ± 1.20 | 40.66 ± 2.21 |
| | | 1.64 | | | | | | | | |
| RSg | 18.63 ± 1.60 | 20.38 ± | 20.89 ± 0.95 | 19.99 ± 0.59 | 20.73 ± 1.22 | 19.48 ± 0.64 | 19.48 ± 0.72 | 19.43 ± 1.20 | 20.96 ± 1.08 | 19.59 ± 0.92 |
| | | 1.32 | | | | | | | | |
| RSa | 15.81 ± 1.42 | 17.96 ± | 19.22 ± 1.40 | 18.90 ± 1.03 | 17.36 ± 1.55 | 18.04 ± 0.87 | 16.53 ± 1.13 | 16.92 ± 0.74 | 18.21 ± 1.15 | 18.77 ± 1.07 |
| | | 1.43 | | | | | | | | |
| Au1 | 16.42 ± 1.46 | 18.08 ± | 17.02 ± 1.26 | 18.48 ± 1.36 | 16.76 ± 0.32 | 17.98 ± 0.68 | 18.60 ± 1.75 | 18.68 ± 0.80 | 17.42 ± 1.46 | 16.91 ± 0.86 |
| | | 1.04 | | | | | | | | |
| DGgd | 34.86 ± 2.50 | 38.63 ± | 38.38 ± 1.53 | 39.21 ± 1.75 | 36.93 ± 2.53 | 36.11 ± 2.06 | 35.22 ± 1.16 | 33.04 ± | 36.51 ± 1.07 | 38.16 ± 1.10 |
| | | 1.47 | | | | | | 2.15† | | |
| CA1pd | 16.98 ± 1.79 | 15.90 ± | 16.80 ± 1.23 | 18.34 ± 0.91 | 16.47 ± 0.98 | 19.18 ± 2.03 | 19.98 ± 1.59 | 19.14 ± 2.06 | 18.37 ± 1.54 | 20.15 ± 1.22 |
| | | 0.97 | | | | | | | | |
| CA2pd | 21.38 ± 1.27 | 18.61 ± | 24.07 ± 1.74 | 20.07 ± 1.71 | 21.47 ± 2.23 | 22.31 ± 1.57 | 21.36 ± 2.52 | 17.72 ± 1.86 | 24.21 ± 1.23 | 20.59 ± 1.35 |
| | | 1.29 | | | | | | | | |
| CA3pd | 31.46 ± 1.68 | 32.94 ± | 34.10 ± 1.52 | 33.99 ± 3.29 | 34.72 ± 1.24 | 33.90 ± 2.52 | 33.71 ± 3.48 | 30.50 ± 1.94 | 33.46 ± 2.04 | 32.81 ± 1.82 |
| | | 2.69 | | | | | | | | |
| dRt | 56.97 ± 2.24 | 48.80 ± | 54.94 ± 1.99 | 51.68 ± 2.34 | 53.38 ± 2.99 | 54.81 ± 1.90 | 50.99 ± 2.22 | 50.92 ± 3.80 | 52.87 ± 1.95 | 52.42 ± 1.19 |
| | | 2.72* | | | | | | | | |
| vRt | 46.12 ± 3.47 | 45.07 ± | 46.91 ± 2.73 | 47.18 ± 2.10 | 52.57 ± 2.00 | 49.70 ± 2.76 | 54.57 ± 3.12 | 46.70 ± 3.99 | 42.97 ± 2.22 | 53.69 ± 2.07 |
| | | 2.26 | | | | | | | | |
| DLStr | 6.98 ± 0.20 | 5.87 ± | 6.55 ± 0.53 | 5.99 ± 0.96 | 7.13 ± 0.70 | 7.46 ± 0.86 | 6.87 ± 0.38 | 6.99 ± 0.43 | 6.84 ± 0.42 | 6.70 ± 0.59 |
| | | 0.44* | | | | | | | | |
| VMStr | 6.80 ± 0.20 | 6.15 ± | 6.75 ± 0.50 | 7.18 ± 0.71 | 7.69 ± 0.35 | 7.23 ± 0.26 | 7.33 ± | 6.88 ± 0.46 | 7.52 ± | 5.99 ± 0.28 |
| | | 0.23 | | | | | 0.24† | | 0.16†† | |
| SN | 12.47 ± 0.55 | 12.82 ± | 11.66 ± 0.91 | 12.98 ± 1.12 | 13.04 ± 1.29 | 15.34 ± 1.58 | 13.27 ± 1.21 | 13.98 ± 0.96 | 13.09 ± 0.44 | 12.54 ± 0.68 |
| | | 1.09 | | | | | | | | |
| AcbC | 8.57 ± 0.53 | 8.48 ± | 7.65 ± 0.40 | 8.40 ± 0.63 | 8.44 ± 0.43 | 8.42 ± 0.53 | 8.59 ± 0.58 | 8.12 ± 0.67 | 8.55 ± 0.25 | 8.27 ± 0.31 |
| | | 0.51 | | | | | | | | |
| AcbS | 10.01 ± 0.67 | 10.62 ± | 10.24 ± 0.64 | 11.55 ± 0.50 | 11.11 ± 0.27 | 11.26 ± 0.67 | 11.42 ± 0.73 | 10.43 ± 0.56 | 11.62 ± 0.84 | 11.95 ± 1.02 |
| | | 0.61 | | | | | | | | |
| MM | 25.86 ± 1.87 | 24.34 ± | 25.02 ± 2.54 | 24.82 ± 0.93 | 20.92 ± 0.93 | 28.12 ± 1.89 | 25.36 ± 2.25 | 25.76 ± 1.27 | 25.24 ± 2.32 | 4.69 ± 2.24 |
| | | 2.53 | | | | | | | | |
| MG | 25.23 ± 1.50 | 26.93 ± | 26.82 ± 1.55 | 26.36 ± 0.95 | 26.75 ± 2.02 | 27.36 ± 1.86 | 26.64 ± 2.22 | 25.50 ± 1.46 | 24.56 ± 1.41 | 25.78 ± 0.77 |
| | | 1.46 | | | | | | | | |

All data represents mean Kv3.1 α-subunit mRNA expression (PSL/mm²) ± SEM (n = 5 − 6)
*p < 0.05, **p < 0.01 (t-test, PCP-vehicle vs vehicle-vehicle); †p < 0.05, ††p < 0.01 (Dunnett's Method, PCP-antipsychotic vs PCP-vehicle).

TABLE 2
Kv3.1 mRNA expression in rat brain after chronic
intermittent exposure to vehicle or PCP (YRING model).

| | Relative Optical Density (ROD) | | |
|---|---|---|---|
| | | PCP | |
| Brain Region | Vehicle | (2.58 mg/kg) | % Control |
| Cortical | | | |
| Regions | | | |
| PrL | 0.112 ± 0.002 | 0.086 ± 0.004** | 77 ± 4** |
| IL | 0.103 ± 0.003 | 0.098 ± 0.004 | 95 ± 4 |
| vO | 0.122 ± 0.003 | 0.122 ± 0.004 | 100 ± 3 |
| lO | 0.101 ± 0.003 | 0.092 ± 0.003 | 91 ± 3 |
| M1 | 0.123 ± 0.004 | 0.120 ± 0.004 | 98 ± 3 |
| M2 | 0.116 ± 0.002 | 0.106 ± 0.004 | 91 ± 3 |
| ACg | 0.126 ± 0.006 | 0.116 ± 0.004 | 92 ± 3 |
| Pir | 0.263 ± 0.006 | 0.243 ± 0.014 | 92 ± 5 |
| RSG | 0.148 ± 0.006 | 0.153 ± 0.008 | 103 ± 5 |
| RSA | 0.127 ± 0.005 | 0.135 ± 0.006 | 106 ± 5 |
| Au1 | 0.138 ± 0.008 | 0.137 ± 0.009 | 99 ± 6 |
| LEnt | 0.096 ± 0.008 | 0.093 ± 0.005 | 97 ± 5 |
| Thalamus | | | |
| AD | 0.215 ± 0.006 | 0.216 ± 0.006 | 100 ± 3 |
| dRt | 0.274 ± 0.007 | 0.258 ± 0.013 | 94 ± 5 |
| vRt | 0.275 ± 0.010 | 0.224 ± 0.008** | 81 ± 3** |
| Hippocampus | | | |
| DG gcl | 0.244 ± 0.005 | 0.239 ± 0.012 | 98 ± 5 |
| CA1pcl | 0.128 ± 0.006 | 0.122 ± 0.007 | 95 ± 5 |
| CA2pcl | 0.156 ± 0.009 | 0.156 ± 0.010 | 100 ± 6 |
| CA3pcl | 0.202 ± 0.007 | 0.203 ± 0.009 | 100 ± 4 |

Data expressed as mean ± SEM relative optical density (ROD) (n = 8 − 10 per treatment group).
**p < 0.01 compared to vehicle at appropriate time point (student t-test, corrected for multiple comparisons by application of the sequential Bonferroni correction factor).

TABLE 3
Kv3.2 mRNA expression in rat brain after chronic
intermittent exposure to vehicle or PCP (YRING model).

| | Relative Optical Density (ROD) | | |
|---|---|---|---|
| | | PCP | |
| Brain Region | Vehicle | (2.58 mg/kg) | % Control |
| Cortical | | | |
| Regions | | | |
| PrL | 0.079 ± 0.002 | 0.092 ± 0.003* | 116 ± 4 |
| IL | 0.079 ± 0.003 | 0.083 ± 0.002 | 105 ± 2 |
| vO | 0.074 ± 0.002 | 0.078 ± 0.003 | 105 ± 4 |
| lO | 0.069 ± 0.002 | 0.073 ± 0.002 | 106 ± 3 |
| M1 | 0.067 ± 0.002 | 0.076 ± 0.003* | 113 ± 4 |
| M2 | 0.076 ± 0.003 | 0.079 ± 0.004 | 104 ± 5 |
| ACg | 0.078 ± 0.002 | 0.075 ± 0.002 | 96 ± 2 |
| Pir | 0.140 ± 0.005 | 0.138 ± 0.006 | 98 ± 4 |
| RSG | 0.065 ± 0.004 | 0.069 ± 0.002 | 106 ± 3 |
| RSA | 0.070 ± 0.002 | 0.065 ± 0.002 | 93 ± 3 |
| Au1 | 0.070 ± 0.002 | 0.068 ± 0.003 | 97 ± 4 |
| LEnt | 0.051 ± 0.003 | 0.048 ± 0.002 | 94 ± 4 |
| Thalamus | | | |
| AV | 0.275 ± 0.006 | 0.269 ± 0.005 | 98 ± 2 |
| AM | 0.248 ± 0.005 | 0.227 ± 0.006* | 91 ± 2 |
| MD | 0.211 ± 0.008 | 0.187 ± 0.010 | 89 ± 5 |
| VA | 0.240 ± 0.008 | 0.223 ± 0.006 | 93 ± 2 |
| VM | 0.206 ± 0.009 | 0.201 ± 0.005 | 98 ± 2 |
| VL | 0.211 ± 0.010 | 0.208 ± 0.004 | 98 ± 2 |
| LDVL | 0.194 ± 0.009 | 0.197 ± 0.004 | 102 ± 2 |
| LDDM | 0.203 ± 0.008 | 0.205 ± 0.007 | 101 ± 3 |
| VPL | 0.175 ± 0.007 | 0.180 ± 0.005 | 103 ± 3 |
| MGD | 0.175 ± 0.006 | 0.164 ± 0.006 | 94 ± 3 |
| MGV | 0.193 ± 0.007 | 0.185 ± 0.003 | 96 ± 2 |
| DLG | 0.195 ± 0.012 | 0.222 ± 0.007 | 114 ± 4 |
| Hippocampus | | | |
| DG gcl | 0.097 ± 0.004 | 0.095 ± 0.007 | 98 ± 7 |
| CA1pcl | 0.069 ± 0.004 | 0.075 ± 0.004 | 109 ± 6 |
| CA2pcl | 0.080 ± 0.002 | 0.082 ± 0.003 | 102 ± 4 |
| CA3pcl | 0.142 ± 0.005 | 0.134 ± 0.005 | 94 ± 4 |

Data expressed as mean ± SEM relative optical density (ROD) (n = 8 − 10 per treatment group).
*p < 0.05 compared to vehicle at appropriate time point (student t-test, corrected for multiple comparisons by application of the sequential Bonferroni correction factor).

TABLE 4
Kv3.3 mRNA expression in rat brain after chronic
intermittent exposure to vehicle or PCP (YRING model).

| | Relative Optical Density (ROD) | | |
|---|---|---|---|
| | | PCP | |
| Brain Region | Vehicle | (2.58 mg/kg) | % Control |
| Cortical Regions | | | |
| PrL | 0.057 ± 0.002 | 0.067 ± 0.003* | 118 ± 5 |
| IL | 0.053 ± 0.002 | 0.059 ± 0.002 | 111 ± 4 |
| vO | 0.073 ± 0.003 | 0.083 ± 0.003* | 114 ± 4 |
| lO | 0.053 ± 0.002 | 0.058 ± 0.002 | 109 ± 4 |
| M1 | 0.085 ± 0.003 | 0.093 ± 0.002 | 109 ± 2 |
| M2 | 0.073 ± 0.002 | 0.075 ± 0.003 | 103 ± 4 |
| ACg | 0.068 ± 0.003 | 0.069 ± 0.003 | 101 ± 4 |
| Pir | 0.111 ± 0.005 | 0.115 ± 0.006 | 104 ± 5 |
| RSG | 0.087 ± 0.004 | 0.092 ± 0.004 | 106 ± 4 |
| RSA | 0.066 ± 0.003 | 0.067 ± 0.003 | 102 ± 4 |
| Au1 | 0.075 ± 0.002 | 0.076 ± 0.003 | 101 ± 4 |
| LEnt | 0.042 ± 0.002 | 0.047 ± 0.002 | 112 ± 5 |
| Thalamus | | | |
| AD | 0.173 ± 0.009 | 0.209 ± 0.001* | 121 ± 1 |
| dRt | 0.186 ± 0.001 | 0.198 ± 0.006 | 106 ± 3 |
| vRt | 0.121 ± 0.010 | 0.161 ± 0.007* | 133 ± 6 |
| Hippocampus | | | |
| DG gcl | 0.254 ± 0.009 | 0.275 ± 0.011 | 108 ± 4 |
| CA1 pcl | 0.144 ± 0.006 | 0.146 ± 0.006 | 101 ± 4 |
| CA2 pcl | 0.149 ± 0.005 | 0.148 ± 0.007 | 99 ± 5 |
| CA3 pcl | 0.204 ± 0.006 | 0.210 ± 0.006 | 103 ± 3 |

Data expressed as mean ± SEM relative optical density (ROD) (n = 8 − 10 per treatment group).
*p < 0.05 compared to vehicle at appropriate time point (student t-test, corrected for multiple comparisons by application of the sequential Bonferroni correction factor).

Abbreviations:

PrL, prelimbic cortex;

IL, infralimbic cortex;

M1, primary motor cortex;

M2, secondary motor cortex;

vO, ventral orbital cortex;

lO, lateral orbital cortex;

Acg, anterior cingulate cortex;

Pir, piriform cortex;

RSg, granular retrosplenial cortex;

Rsa, agranular retrosplenial cortex;

Aul, primary auditory cortex;

LEnt, lateral entorhinal cortex;

DGgcl, granule cell layer of the dentate gyrus;

CA1-CA3 pcl, pyramidal cell layer of the CA1-CA3 fields of

the hippocampus;

AD, anterodorsal nucleus of the thalamus;

AV, anteroventral nucleus of the thalamus;

AM, ateromedial nucleus of the thalamus;

MD, mediodorsal nucleus of the thalamus;

VA, ventral anterior nucleus of the thalamus;

VM, ventral medial nucleus of the thalamus;

VL, ventral lateral nucleus of the thalamus;

LDVL, laterodorsal thalamic nucleus, ventrolateral part;

LDDM, laterodorsal thalamic nucleus, dorsomedial part;

VPL, ventral posterolateral nucleus of the thalamus;

MGD, medial genicluate nucleus, dorsal part;

MGV, medial genicluate nucleus, ventral part;

DLG, dorsal lateral geniculate nucleus

dRt, vRt, dorsal and ventral parts of the reticular nucleus of the thalamus.

DLStr, dorsolateral striatum;

VMStr, ventromedial striatum;

SN, substantia nigra;

AcbC, nucleus accumbens core;

AcbS, nucleus accumbens shell;

MM, mammillary body;

MG, medial geniculates.

The decrease in Kv3.1 α-subunit mRNA expression observed in these studies parallel the selective changes previously observed in parvalbumin mRNA expression within the prelimbic cortex and the reticular nucleus of the thalamus. Moreover, the PCP-induced decreases in Kv3.1 α-subunit mRNA expression were reversed by chronic co-administration of Y-931 and olanzapine, as were PCP-induced changes in parvalbumin mRNA expression. Since parvalbumin expression is altered within the prefrontal cortex of schizophrenic patients and in rats after treatment with chronic PCP, these data suggest that Kv3.1 α-subunit mRNA may also be altered within schizophrenia in a similar manner.

As the chronic model of schizophrenia also produces an increase in Kv3.2 and Kv3.3 mRNA expression in the rat, these data suggest that Kv3.2 and Kv3.3 mRNA may also be altered in schizophrenia.

Drugs acting at the Kv3 channels have potential therapeutic roles in Schizophrenia. The hypothesis is that the decrease in Kv3.1 mRNA may affect the firing activity of the GABAergic interneurons in which it is located, which further contributes to an imbalance between the excitatory (glutamatergic) and inhibitory (GABAergic) pathways within the prefrontal cortex (which may be linked to negative symptoms and cognitive dysfunction). The changes in Kv3.2 mRNA suggest that thalamic relay neurons may also be mis-firing, which again may contribute to an imbalance between the excitatory (glutamatergic) and inhibitory (GABAergic) pathways within the prefrontal cortex. The changes in Kv3.3 mRNA may also disrupt the balance between the excitatory (glutamatergic) and inhibitory (GABAergic) pathways within the prefrontal cortex.

A drug which can restore Kv3.1, Kv3.2 and Kv3.3 expression may restore GABAergic interneuron activity back to normal.

Kv3.1 may be a good marker for the screening of antipsychotic activity because of the reversal with the a typical antipsychotic olanzapine and Y-931 as shown in these data.

This application is based on application No.02007114.8 filed in EPC, the contents of which are incorporated hereinto by reference.

1. A method for diagnosing schizophrenia, which comprises use of at least one kind of polynucleotide selected from the group consisting of a polynucleotide having SEQ ID NO:1, a polynucleotide having SEQ ID NO:3, a polynucleotide having SEQ ID NO:5, a fragment thereof, and a polynucleotide having a sequence complementary to such sequence, as an index of schizophrenia.

2. The method of claims 1, wherein the at least one kind of polynucleotide is selected from the group consisting of a polynucleotide having SEQ ID NO:1, a polynucleotide having SEQ ID NO:3, a polynucleotide having SEQ ID NO:5 and a polynucleotide having a sequence complementary to such sequence.

3. The method of claim 1, wherein the at least one kind of polynucleotide is a polynucleotide having SEQ ID NO:1.

4. A method for diagnosing schizophrenia, which comprises use of at least one kind of polypeptide selected from the group consisting of a polypeptide encoded by the polynucleotide of SEQ ID NO:1, a polypeptide encoded by the polynucleotide of SEQ ID NO:3, a polypeptide encoded by the polynucleotide of SEQ ID NO:5, and a fragment thereof, as an index of schizophrenia.

5. The method of claim 4, wherein the at least one kind of polypeptide is selected from the group consisting of a polypeptide encoded by the polynucleotide of SEQ ID NO:1, a polypeptide encoded by the polynucleotide of SEQ ID NO:3 and a polypeptide encoded by the polynucleotide of SEQ ID NO:5.

6. The method of claim 4, wherein the at least one kind of polypeptide is a polypeptide encoded by the polynucleotide of SEQ ID NO:1.

7. A method for identifying a compound modulating the activity of Kv3 channels, which comprises analyzing an expression of Kv3 channel using at least one kind of polynucleotide selected from the group consisting of a polynucleotide having SEQ ID NO:1, a polynucleotide having SEQ ID NO:3, a polynucleotide having SEQ ID NO:5, a fragment thereof and a polynucleotide having a sequence complementary to such sequence.

8. The method of claim 7, wherein the expression of Kv3 channel is analyzed using at least one kind of polynucleotide selected from the group consisting of a polynucleotide having SEQ ID NO:1, a polynucleotide having SEQ ID NO:3, a polynucleotide having SEQ ID NO:5 and a polynucleotide having a sequence complementary to such sequence.

9. The method of claim 7, wherein the expression of Kv3 channel is analyzed using a polynucleotide having SEQ ID NO:1.

10. A method for identifying a compound modulating the activity of Kv3 channels, which comprises analyzing an expression of Kv3 channel using at least one kind of polypeptide selected from the group consisting of a polypeptide encoded by the polynucleotide of SEQ ID NO:1, a polypeptide encoded by the polynucleotide of SEQ ID NO:3, a polypeptide encoded by the polynucleotide of SEQ ID NO:5, and a fragment thereof.

11 The method of claim 10, wherein the expression of Kv3 channels is analyzed using at least one kind of polypeptide selected from the group consisting of a polypeptide encoded by the polynucleotide of SEQ ID NO:1, a polypeptide encoded by the polynucleotide of SEQ ID NO:3, and a polypeptide encoded by the polynucleotide of SEQ ID NO:5.

12. The method of claim 10, wherein the expression of Kv3 channel is analyzed using a polypeptide encoded by the polynucleotide of SEQ ID NO:1.

13. A method for screening a compound regulating the expression of at least one kind of polynucleotide selected from the group consisting of a polynucleotide having SEQ ID NO:1, a polynucleotide having SEQ ID NO:3 and a polynucleotide having SEQ ID NO:5, which comprises the steps of
(a) bringing a test compound into contact with a cell (as used herein, the cell is capable of expression of polynucleotide, which is at least one kind of polynucleotide selected from the group consisting of a polynucleotide having SEQ ID NO:1, a polynucleotide having SEQ ID NO:3, a polynucleotide having SEQ ID NO:5 and a fragment thereof, and whose expression is desired to be controlled;
(b) detecting the expression of polypeptide encoded by the polynucleotide in the cell; and
(c) determining a compound promoting or suppressing the expression of the polypeptide as compared to a control (administration of vehicle alone).

14. A method for screening a compound regulating the expression of a polynucleotide having SEQ ID NO:1, which comprises the steps of:
(a) bringing a test compound into contact with a cell (as used herein, the cell is capable of expression of a polynucleotide having SEQ ID NO:1);
(b) detecting the expression of polypeptide encoded by the polynucleotide in the cell; and
(c) determining a compound promoting or suppressing the expression of the polypeptide as compared to a control (administration of vehicle alone).

15. A method for screening a compound regulating the expression of at least one kind of polynucleotide selected from the group consisting of a polynucleotide having SEQ ID NO:1, a polynucleotide having SEQ ID NO:3 and a polynucleotide having SEQ ID NO:5, which comprises the steps of:
(a) bringing a test compound into contact with a cell (as used herein, the cell is capable of expression of polynucleotide, which is at least one kind of polynucleotide selected from the group consisting of a polynucleotide having SEQ ID NO:1, a polynucleotide having SEQ ID NO:3, and a polynucleotide having SEQ ID NO:5, and whose expression is desired to be controlled);
(b) detecting the expression of polynucleotide in the cell; and
(c) determining a compound promoting or suppressing the expression of the polynucleotide as compared to a control (administration of vehicle alone).

16. A method for screening a compound regulating the expression of a polynucleotide having SEQ ID NO:1, which comprises the steps of:
(a) bringing a test compound into contact with a cell (as used herein, the cell is capable of expression of polynucleotide having SEQ ID NO:1);
(b) detecting the expression of polynucleotide in the cell; and
(c) determining a compound promoting or suppressing the expression of the polynucleotide as compared to a control (administration of vehicle alone).


SEQUENCE LISTING (9 sequences)

SEQ ID NO: 1 - DNA, 3977 bp, Rattus norvegicus
  CDS (1162)..(2919)
  gene (1)..(3977)
  polyA_signal (3958)..(3963)
  ref <301>: AUTHORS: Luneau,C.J., Williams,J.B., Marshall,J., Levitan,E.S., Oliva,C., Smith,J.S., Antanavage,J., Folander,K., Stein,R.B., Swanson,R., Kaczmarek,L.K. and Buhrow,S.A.
  ref <302>: TITLE: Alternate splicing contributes to K+ channel diversity in the mammalian central nervous system.
  ref <303>: JOURNAL: Proc. Natl. Acad. Sci. USA.
  ref <304>: VOLUME: 88
  ref <305>: ISSUE: 5
  ref <306>: PAGES: 3932-3936
  ref <308>: DATABASE ACCESSION NUMBER: NM_012856
  ref <309>: DATABASE ENTRY DATE: 1991-05-01
gaacgagcaagcgagcaggcagcggagcagcagcccggaggcagcagcggcggcggtggtggcggcagtggccgctccgcgcctcgcctccctggcctcgccagcctcgttgcgccgcgcccggacctgccacccccgcctaccgggcccgacctcggcagcgcccctcgcatcggctgccagcgccacccgccctcccagagcagtgagcggagtgcggggcgcagaaacaccggagccagtgcccgccagccccgccaagcaaaacgggctgagagaacctacgctcggcccagcgcggctccacagccgtccgggagctgtcccttcagcaccgccgcggaagcctgagtcccagcgcagcccagcggaacccctgacccagcccgagcgcaccaggcgctgcgcttagcggcagccccgcggggagcacccttgtgcccctacccaggggacctgccatctcctccgggaggcggggagaaggaacgaggagggaggtcgggcagcttcacccgccaccgaggggagtaggcagacaagcaaggagatccgaaggggatcggtcacgcagggaggagagcaagccgcatccgtccccaccgcaccgggagcgcgctgcggcgaccaccgcagagccaagcgcggatttctcgtaaccccctctttcctagcaggggctggtctatcggtccggcccccgaaacggagcagctgaccttcccctaaaaagctttgtgtcgatttctccatttttcccgctgagatggtaccgggagcccggcccccccatcatctctcccctcctcccgggcctcgccgctgagcgccgccccactcccttcctccctccctctttacctccctcctctcttccactgcctcctccgcagccaggccagctccctctcacactaggcgccttaaagaccctaaggagcccgcgcacacacccctggacccgcacctgactgacagctccccctgagggggcttggctcgttggttggggtgggtggccagagccgcggtcctctgtgccccccgacggctggggggaggggggaagaggccctgcgcccccctccccgtcgccaactccccctggcggcagctcccatgggtgtcgctgggccgcgccatgcctaagggggcgccgcgatgggccaaggggacgagagcgagcgcatcgtgatcaacgtgggcggcacgcgccaccagacgtaccgctcgacgctgcgcacgctgcccggcacgcggcttgcctggctggcggagccggacgcccacagccacttcgactatgacccgcgcgctgacgagttcttcttcgaccgccacccgggcgtcttcgctcacatcctgaactattaccgcaccggcaagctacactgcccggccgacgtgtgcgggccgctctacgaggaggagctggccttctggggcatcgacgagacggacgtggagccctgctgctggatgacgtatcgccagcaccgcgacgctgaggaggcgctggacagctttggtggcgcgcccttggacaacagcgccgacgacgcggacgccgacggccccggcgactcgggcgacggcgaggacgagctggagatgaccaagagattggcgctcagtgactccccagatggccggcctggcggcttctggcgccgctggcagccacgcatctgggcactgttcgaggacccctactcatcccgctatgcgcggtacgtggcctttgcctccctcttcttcatcctggtctccatcacaaccttctgtctggagacccacgagcgcttcaaccccatcgtgaacaagacagaaattgagaacgttcgaaatggcacacaagtgcggtactaccgggaagcggagacggaggccttcctcacctacatcgagggtgtctgcgtggtctggttcaccttcgagttcctcatgcgtgttgtcttctgtcccaacaaggtggagttcatcaagaactccctcaatatcattgacttcgtggccatcctccccttctacctggaggtgggcctaagtggcctgtcctccaaagctgccaaggacgtgctaggcttcctgcgcgtcgtgcgcttcgtgcgcatcctccgtatcttcaagctgacccgccattttgtgggcctgcgggtcctgggccacacgctccgtgccagcaccaacgagttcctgctgcttatcatcttcctggccctgggtgtgctcatctttgccaccatgatctactatgctgagaggataggggcacagcccaatgaccccagcgccagcgaacacacacactttaaaaatatccccatcggcttctggtgggctgtggtcaccatgacgacactgggctatggagacatgtatcctcagacgtggtctgggatgttggtgggagcactgtgcgccctggctggtgtgctgaccattgccatgccggtgcccgtcatcgtgaacaattttgggatgtactactctttagccatggctaagcagaaactaccaaagaaaaaaaagaagcatattccgcggccaccacagctgggatctcccaattattgtaaatctgtcgtaaactctccacaccacagtactcagagtgacacatgtccgctggcccaggaagaaattttagaaattaacagagcagattccaaactgaatggggaggtggcgaaggccgcgctggcgaacgaagactgcccccacatagaccaggccctcactcccgatgagggcctgccctttacccgctcgggcacccgcgagagatacggaccctgcttcctcttatcaaccggggagtacgcgtgcccgcctggtggaggaatgagaaaggatctttgcaaagaaagccctgtcattgctaagtatatgccgacagaggctgtgagagtgacttgaccaggcggcttggccgaggacactggtggctattaagcatctgggtggacctgcagcccctcctcaccctcggacagagtaaattcacgccatgcaggtttgccggacgagtccgagtggccccgggcattgtactaagacggacgtagctttttctacggcccacggtaactgaccgtagaggattcgcccttctttcttttgattttctttttctttcttttttttttttttagaagttaccttttatttggggagggggggtcaaggggagccttagcatgacttgcatgaagttaaacagaaaacccagctaaaccaaccccaccagcccctgctgtgactgtatgctcactctaaccaccaccgagaaccagagaaaggcaagagtctcttatttcttccagcattctttgacacgtagtgtcggagtcaaactgtgacggcgcttggtagaaccctgtacatttccgggagtgggtgcgggggggggggggaggtcagggactaaggatggggccaggggagtggattgcttttttgtacacgagacccagaggaaaggtcccaaaaaggcagtcagtcatcggtcacattgacctcaccttcatagctcacctctcgctcggaaaaccaagaactatgctccgaataggattgtggaaattcacactgccaccctcttccacctccgtttggcatgcttgcgacccaatggacctctccccaagaccccgacagcggctagtttaggtcaattctctcatcttggtgtgtagctccagtgtgaccaagtttcataacaaattgtttcatagttacaccacatggattttccaatgtcattttaaagccaggatgtagagtccctccagttcaggaccacccagaggggcgcctcccctccacctttactaaggcgaaaaccgacactgtatgcaatttagtacttcagagctcaaagaaaaaaaaaaaaagaaaaaaaaagaaaaaagaaaaactgcatgcccaatgttatgcaaagagattcgagcatgaaataaattttgtaacatggt

SEQ ID NO: 2 - protein, 585 aa, Rattus norvegicus
MGQGDESERIVINVGGTRHQTYRSTLRTLPGTRLAWLAEPDAHSHFDYDPRADEFFFDRHPGVFAHILNYYRTGKLHCPADVCGPLYEEELAFWGIDETDVEPCCWMTYRQHRDAEEALDSFGGAPLDNSADDADADGPGDSGDGEDELEMTKRLALSDSPDGRPGGFWRRWQPRIWALFEDPYSSRYARYVAFASLFFILVSITTFCLETHERFNPIVNKTEIENVRNGTQVRYYREAETEAFLTYIEGVCVVWFTFEFLMRVVFCPNKVEFIKNSLNIIDFVAILPFYLEVGLSGLSSKAAKDVLGFLRVVRFVRILRIFKLTRHFVGLRVLGHTLRASTNEFLLLIIFLALGVLIFATMIYYAERIGAQPNDPSASEHTHFKNIPIGFWWAVVTMTTLGYGDMYPQTWSGMLVGALCALAGVLTIAMPVPVIVNNFGMYYSLAMAKQKLPKKKKKHIPRPPQLGSPNYCKSVVNSPHHSTQSDTCPLAQEEILEINRADSKLNGEVAKAALANEDCPHIDQALTPDEGLPFTRSGTRERYGPCFLLSTGEYACPPGGGMRKDLCKESPVIAKYMPTEAVRVT

SEQ ID NO: 3 - DNA, 2441 bp, Rattus norvegicus
  gene (1)..(2411)
  CDS (37)..(1953)
  ref <301>: AUTHORS: McCormack,T., Vega-Saenz de Miera,E.C. and Rudy,B.
  ref <302>: TITLE: Molecular cloning of a member of a third class of Shaker-family K+channel genes in mammals
  ref <303>: JOURNAL: Proc. Natl. Acad. Sci. U.S.A.
  ref <304>: VOLUME: 88
  ref <305>: ISSUE: 9
  ref <308>: DATABASE ACCESSION NUMBER: NM_139217
  ref <309>: DATABASE ENTRY DATE: 1991-05-01
gtgttcgccttccctagtcatgtctgagccacagagatgggcaagatcgagaacaacgagagggtgatcctcaatgtcggaggcaccaggcacgaaacctaccgcagcactctcaagacccttcctggaactcgcctggcccttctcgcctcctctgaacctcagggcgactgcctgactgctgcgggtgacaagctgcagccgctgccccctccgctgtctccaccgccgcgaccgcctcccttgtcccctgtccccagcggctgcttcgagggcggcgcaggcaactgcagttcgcacggtggcaatggcagcgaccaccctgggggaggccgcgaattcttcttcgatcgccacccaggagtcttcgcctatgtgctcaactactaccgcacgggcaagctgcactgccccgccgacgtgtgtggaccgctcttcgaggaagagctggcattctggggcatcgatgagaccgacgtggagccctgctgctggatgacctacaggcagcaccgggacgcggaggaggccctggatatcttcgagacacccgacctcatcggaggcgaccctggtgatgatgaggacctagggggcaagagactgggcattgaggatgctgcggggctgggaggacccgatggcaagtctggccgctggaggaagctgcagcctcgcatgtgggctctctttgaggacccctattcatccagagccgctaggtttattgcttttgcttctctgttcttcattttggtttccatcacaaccttttgcctggagacacacgaagctttcaatattgttaaaaacaagacagagccagtcatcaacggcaccagcgctgttctccagtatgaaatcgaaacggatcctgccttgacatatgtggaaggagtgtgtgtggtgtggtttacttttgaatttttagtccgtattgttttctcgcccaataaacttgagttcatcaaaaatctattgaacatcattgactttgtggccatcctccccttctacttagaggtgggactcagcgggctgtcttccaaagcggctaaagatgtgctcggctttctcagggtggttaggtttgtgaggatcctgagaatcttcaagcttacccgccatttcgtaggtctgagagtgctcggacacactcttcgtgcgagcaccaatgaatttttgttgctgatcatctttctggctctgggagttttgatattcgctacgatgatctactacgctgagcgagtaggggctcaacctaatgatccctcagcgagtgagcacacacagttcaaaaacatccccattggtttctggtgggctgtggtgaccatgactaccttaggctatggggatatgtacccccaaacatggtcagggatgttggtgggggccttgtgtgctctggctggagtgctgaccatagctatgcctgtgcccgtcattgtcaacaattttgggatgtactactccttggcaatggcgaagcagaaacttccaagaaaaagaaagaagcacattcctcctgcccctctggcaagctcacctacattttgcaagacagaattaaacatggcttgtaacagtacccagagtgacacatgtctgggcaaagaaaaccggcttctggaacataacagatcagtgttatcaggtgacgacagtacaggaagtgagccgccattatcacctccggaaaggctccccatcagacgctctagtaccagagacaaaaacagaagaggggaaacatgtttcctgttgacgacaggtgattacacgtgcgcttctgatggaggaatcaggaaaggatatgaaaaatcccgaagcttaaacaacatagcgggcttggcaggcaatgctctgagactctctccagtaacatccccctacaactctccgtgtcctctgaggcgctctcggtctcccatcccatctatcttgtaaaccaaactgcaccaatcggctaaattctatcactacaactcctgtccaccctgtactggaaacaattaactatagagtttctccaggctccattaagaacagtgtagatcctgcatgacattactattcgaagtcagaaatgctactcgagtagctagaacatcttttcctggctttaaagatggcctaaagtagtgctgctactcagtaagagttgcccaatttcctttcgctatacggtgaccaatagcgtcagatattggccagtgcacgaatgcataagcaaatcttcagggagatattctttaatagtgtgcttctaaatgccaaccacttcactggaccttcctttcttgtgactgactccaacccattctgaagatgtctgggatcctatctagatcgtataaaccatgactttggtcagctcgtttgcatggaccatcttgcctgtatcacctgaatacagtgtgtagcgtcctggtacc

SEQ ID NO: 4 - protein, 638 aa, Rattus norvegicus
MGKIENNERVILNVGGTRHETYRSTLKTLPGTRLALLASSEPQGDCLTAAGDKLQPLPPPLSPPPRPPPLSPVPSGCFEGGAGNCSSHGGNGSDHPGGGREFFFDRHPGVFAYVLNYYRTGKLHCPADVCGPLFEEELAFWGIDETDVEPCCWMTYRQHRDAEEALDIFETPDLIGGDPGDDEDLGGKRLGIEDAAGLGGPDGKSGRWRKLQPRMWALFEDPYSSRAARFIAFASLFFILVSITTFCLETHEAFNIVKNKTEPVINGTSAVLQYEIETDPALTYVEGVCVVWFTFEFLVRIVFSPNKLEFIKNLLNIIDFVAILPFYLEVGLSGLSSKAAKDVLGFLRVVRFVRILRIFKLTRHFVGLRVLGHTLRASTNEFLLLIIFLALGVLIFATMIYYAERVGAQPNDPSASEHTQFKNIPIGFWWAVVTMTTLGYGDMYPQTWSGMLVGALCALAGVLTIAMPVPVIVNNFGMYYSLAMAKQKLPRKRKKHIPPAPLASSPTFCKTELNMACNSTQSDTCLGKENRLLEHNRSVLSGDDSTGSEPPLSPPERLPIRRSSTRDKNRRGETCFLLTTGDYTCASDGGIRKGYEKSRSLNNIAGLAGNALRLSPVTSPYNSPCPLRRSRSPIPSIL

SEQ ID NO: 5 - DNA, 3410 bp, Rattus norvegicus
  CDS (223)..(2262)
  gene (1)..(3410)
  ref <301>: AUTHORS: Vega-Saenz de Miera,E., Moreno,H., Fruhling,D., Kentros, C. and Rudy,B.
  ref <302>: TITLE: Cloning of ShIII (Shaw-like) cDNAs encoding a novel high-voltage-activating, TEA-sensitive, type-A K+ channel
  ref <303>: JOURNAL: Proc. R. Soc. Lond., B, Biol. Sci.
  ref <304>: VOLUME: 248
  ref <305>: ISSUE: 1321
  ref <306>: PAGES: 9-18
  ref <308>: DATABASE ACCESSION NUMBER: NM_053997
  ref <309>: DATABASE ENTRY DATE: 1991-05-01
aagctttttgtgcaatcccacctcctctagctaggcttggttagagtctgcctagccgtggccccgcccactccatcagtccaatcagctcgtgttctccctccacctgcccaatcactccctccccatccattcatcgcctcctcctgcccaatcacatctgcttctctaccaaggctccgccccaacaccctgcccccctcagccccgcctccctgtccaatgctcagctcagtgtgcgtctggtcgttcagcgggcgccaggggacccgcaagcagcattctcagccggcgccaacgccgcagccgcctgagtcctcaccgccgcctctgctaccgccgccgcagcagcagtgcgctcagcccggcaccgccgcctccccggcgggtgccccgctttcctgtgggcctgggggccggcgcgccgagccatgctccgggctgccggcggtggccatggggcggcacggcggcggcggcggcgacagcggtaagatcgtgatcaacgtgggcggcgtgcgccatgagacgtaccgctccacgttgcgcaccctaccggggacccggctggccgggctgaccgagcccgaggcggccgcgcgctttgactacgacccgggcacggacgagttcttctttgaccgtcacccgggcgtcttcgcctacgtgctcaactactaccgcaccggcaagctgcactgcccggccgacgtgtgcgggccgctcttcgaggaggagctgggcttctggggcatagacgagacggacgtggaggcctgctgctggatgacctaccgccagcaccgtgatgccgaagaggcactggactccttcgaggctccggactcctcgggcaacgccaacgccaacgccggaggcgcccatgatgcgggcctggacgacgaggcgggcgcaggaggtggcggcctggacggggcaggcggcgagctcaagcgtctgtgttttcaggacgcgggcggaggtgccggaggacctgccggaggcccgggcggcgcgggcggcacgtggtggaggcgctggcagccccgcgtgtgggcgctttttgaggacccctactcgtcgcgggccgccaggtacgtggccttcgcctccctattctttatcctcatctccatcaccaccttctgcctggagacacacgagggcttcatccacatcagcaacaagacggtgacgcaggcctctccaatccctggggctcccccggagaatatcaccaatgtggaggtggagacggaaccctttttgacctacgtggaaggcgtgtgtgtggtctggttcacctttgagtttctcatgcgggtcaccttctgcccggataaggtggagtttctcaaaagcagtcttaacatcatcgactgtgtagccatcttgcccttctacttggaagtgggcctgtcaggtctcagctccaaagctgccaaggacgtgctgggcttcctgcgcgtcgtccgcttcgtgcgcatcctccgtatcttcaagctgacccgccattttgtgggcctgcgggtcctgggccacacgctccgtgccagcaccaacgagttcttgttactcattattttcctggctctgggggtcctcatctttgccaccatgatctactatgccgagcgcatcggggccgatcctgatgacatcctgggctccaaccacacctacttcaagaacatccccatcggcttctggtgggctgtggtcaccatgaccacactgggctatggtgacatgtaccccaagacatggtctgggatgctggtcggggcactgtgtgccctggctggtgtgctgaccattgccatgccggtgcccgtcattgtcaacaacttcggcatgtactattcactggctatggccaagcagaaattgccaaagaagaaaaacaaacatatccccaggcccccgcagcctggctcacccaactactgcaagcccgatcccccgcctccgcccccaccacacccccaccacggcagcggtggcatcagcccaccaccgcccatcacccctccttccatgggggtgactgtggctggggcttacccacctggaccccacacgcaccccgggctgcttaggggtggtgctgggggactgggaattatgggattgcctcctctgccagcccctggtgagccctgcccactggctcaagaagaggtgattgaaaccaacagggcaggtgaggctggagccaggactggaggtgtgggaagacctgggggttgggggtcggggtagggggaggaggcggcagggttggaggggatgcagaggcaggatttctcttaggttcaggctgagcagagtggaaggcaacagggcagacagaagggacaagtggctagaaggctggagaggaggcacaaggagccacagaaaccggattccgtgtacaagagagtgtgtgaacatggatttcagccaggctgctctgaaaggcccctggtcagaagcagcacagtgagggtctttggagaagagactcagaaacccaggtgtgatggtgcatgcctcccttcctttgaaggtacttggtacctcacatacttgggagatagaagtaggaggatgacgagttcaaggctagcctgggtgacatgtccctgtctcaaaaatcatgggagaaaaaaatgaaagagaaagaaaggaaagaggttgggaggggtaggggagaaagagaaaggcttggtggcactctcctatagttccaacactggggagactgaggcaggagggtcaacacgtgttttgaggctatcctgggctacatacacagaaacaaaaggtctgagtagcttcctgggggctgctagttcacaccgattcttagggatattcctatttctctatgagaaggcaagggaagacagggagagatagaaaggaaaaagggaaaggaatgagagccacgtatggtaatcctaaaatggaggaggatcaggagttcaaggccggtgtcagctacgtgagaccctgtctcagaaagaaaggggctcctgtaaagcacaggaagtcgcgcgtgactcctggcaaggctagaggcaggtctggctactgtcactttgtgactgtggccggtagtcctgagagccagagctggggaatgtaagtcacttaaggagagagggagaatggagtcaggctgccaggagggtcgaggtggaccctcgtaaaggaaggcaaaagggaggaacagaccaaggccagcatcagggtgcggagctgccaggcagggtctgcacagggcagcaggaggcactagggatcccagagagttcttgtaagagaagaggcagccaggactggttgaacgtacctgttatcccaggatttgggaagctgaggcagaaggacagttaagttcagagctagcctggactatagag

SEQ ID NO: 6 - protein, 679 aa, Rattus norvegicus
MLSSVCVWSFSGRQGTRKQHSQPAPTPQPPESSPPPLLPPPQQQCAQPGTAASPAGAPLSCGPGGRRAEPCSGLPAVAMGRHGGGGGDSGKIVINVGGVRHETYRSTLRTLPGTRLAGLTEPEAAARFDYDPGTDEFFFDRHPGVFAYVLNYYRTGKLHCPADVCGPLFEEELGFWGIDETDVEACCWMTYRQHRDAEEALDSFEAPDSSGNANANAGGAHDAGLDDEAGAGGGGLDGAGGELKRLCFQDAGGGAGGPAGGPGGAGGTWWRRWQPRVWALFEDPYSSRAARYVAFASLFFILISITTFCLETHEGFIHISNKTVTQASPIPGAPPENITNVEVETEPFLTYVEGVCVVWFTFEFLMRVTFCPDKVEFLKSSLNIIDCVAILPFYLEVGLSGLSSKAAKDVLGFLRVVRFVRILRIFKLTRHFVGLRVLGHTLRASTNEFLLLIIFLALGVLIFATMIYYAERIGADPDDILGSNHTYFKNIPIGFWWAVVTMTTLGYGDMYPKTWSGMLVGALCALAGVLTIAMPVPVIVNNFGMYYSLAMAKQKLPKKKNKHIPRPPQPGSPNYCKPDPPPPPPPHPHHGSGGISPPPPITPPSMGVTVAGAYPPGPHTHPGLLRGGAGGLGIMGLPPLPAPGEPCPLAQEEVIETNRAGEAGARTGGVGRPGGWGSG

SEQ ID NO: 7 - DNA, 45 bp, Artificial Sequence
  oligonucleotide (45mer) used for in situ hybridization for the Kv3.1 alpha-subunit mRNA
atagtcgaagtggctgtgggcgtccggctccgccagccaggcaag

SEQ ID NO: 8 - DNA, 45 bp, Artificial Sequence
  oligonucleotide (45mer) used for in situ hybridization for Kv3.2 mRNA
cacatctttagccgctttggaagacagcccgctgagtcccacctc

SEQ ID NO: 9 - DNA, 45 bp, Artificial Sequence
  oligonucleotide (45mer) used for in situ hybridization for Kv3.3 mRNA
agaatgctgcttgcgggtcccctggcgcccgctgaacgaccagac